(19) United States
(12) Patent Application Publication
(10) Pub. No.: US 2011/0124577 A1
(43) Pub. Date: May 26, 2011

(54) PREPARATION FOR ORAL ADMINISTRATION COMPRISES QUERCETIN GLYCOSIDE AND A WATER-BASED SOLVENT EXTRACT OF CARTILAGE CONTAINING CHONDROITIN SULFATE

(75) Inventors: Aki Taimatsu, Mishima-gun (JP); Setsu Ijichi, Mishima-gun (JP); Ayako Kidokoro, Mishima-gun (JP)

(73) Assignee: SUNTORY HOLDINGS LIMITED, Osaka (JP)

(21) Appl. No.: 13/056,359

(22) PCT Filed: Jun. 15, 2009
(86) PCT No.: PCT/JP2009/060857

§ 371 Date: Jan. 28, 2011

(30) Foreign Application Priority Data
Jul. 30, 2008 (JP) 2008-196743

(51) Int. Cl. A61K 38/14; A61K 31/7048; A61P 29/00; A23L 1/22

(52) U.S. Cl. 514/20.9; 514/27; 426/536

(57) ABSTRACT

It is an object of the present invention to provide a preparation for oral administration in which the bitter taste derived from quercetin glycoside is improved.

The bad taste derived from quercetin glycoside can be improved, thereby improving palatability, by blending an aqueous chondroitin sulfate extract in a preparation for oral administration containing quercetin glycoside.

### TECHNICAL FIELD

The present invention relates to a preparation for oral administration containing an aqueous chondroitin sulfate extract and quercetin glycoside. Especially, the present invention relates to a preparation for oral administration in which the bad taste derived from quercetin glycoside is improved. Further, the present invention relates to a method for improving the bad taste of quercetin glycoside.

### BACKGROUND ART

Quercetin, one of flanoids, which is contained in plants such as onions and buckwheat in a large amount, is known to exhibit various physiological effects. Quercetin and a glycoside thereof have been reported to exhibit effects such as an anti-inflammatory effect, an antioxidant effect, a vasoconstriction effect, and a capillary wall strengthening effect, and are used in applications such as foods, pharmaceuticals, and cosmetics. In addition, quercetin has been reported to inhibit some enzymes involved in cell growth. However, quercetin glycoside has a unique taste, especially a bitter taste.

However, as a measure to deal with arthritis and joint pain, a method in which a component derived from a natural product, such as chondroitin sulfate (e.g., shark cartilage powder) and glucosamine, is ingested has been investigated as a very safe method for the treatment, prevention, and alleviation of arthritis (Non Patent Literature 1).

### CITATION LIST

### Non Patent Literature

NPL 1: “Functional Glyco-Materials: Their Development and Application to Foods” (Kuniyo Inoue Supervising Editor, 2005, CMC Publishing Co., Ltd., pp. 394-400)

### SUMMARY OF INVENTION

### Technical Problem

The taste and palatability of quercetin glycoside often are problematic for oral ingestion due to its unique taste, especially its bitter taste. Especially, since the taste derived from quercetin glycoside is suppressed to facilitate continuous ingestion or long-term ingestion when oral ingestion of quercetin glycoside is attempted on a daily basis as a functional food such as a supplement, development of a preparation for oral administration in which the taste peculiar to quercetin glycoside is suppressed is required. It is an object of the present invention to provide a method for suppressing the taste derived from quercetin glycoside, and an oral preparation in which the taste derived from quercetin glycoside is suppressed.

### Solution to Problem

As a result of research concerning the above problems, the present inventors discovered that the bitter taste of quercetin glycoside could be effectively masked by an aqueous chondroitin sulfate extract, thereby completing the present invention.

In one aspect, the present invention is a preparation for oral administration containing an aqueous chondroitin sulfate extract and quercetin glycoside. Especially, in view of the fact that the preparation is required to be orally ingested on a daily basis in food uses such as, as a supplement, the preparation for oral administration according to the present invention effectively suppresses the taste of quercetin glycoside, and is thus suitable.

In a separate aspect, the present invention is a method for producing a preparation for oral administration containing quercetin glycoside. This method includes a step of blending quercetin glycoside with an aqueous chondroitin sulfate extract.

In another aspect, the present invention is a method for improving the taste of a composition containing quercetin glycoside. This method includes a step of adding an aqueous chondroitin sulfate extract.

More specifically, the present invention includes, but is not limited to, the following inventions.

1. A preparation for oral administration comprising an aqueous chondroitin sulfate extract and quercetin glycoside.
2. The preparation for oral administration according to the above-described 1, wherein the quercetin glycoside is enzyme-treated rutin.
3. The preparation for oral administration according to the above-described 1 or 2, further comprising glucosamine.
4. The preparation for oral administration according to any one of the above-described 1 to 3, wherein the preparation is a solid preparation.
5. The preparation for oral administration according to the above-described 4, wherein the solid preparation is a tablet or a granule.
6. The preparation for oral administration according to any one of the above-described 1 to 5, wherein a weight ratio of the aqueous chondroitin sulfate extract and the quercetin glycoside is 1:50 to 200:1.
7. The preparation for oral administration according to any one of the above-described 1 to 6, wherein the aqueous chondroitin sulfate extract is a chondroitin sulfate that exists as a complex with a protein.
8. The preparation for oral administration according to any one of the above-described 1 to 7, wherein the preparation is a functional food.
9. A method for producing a preparation for oral administration comprising quercetin glycoside, the method comprising blending quercetin glycoside with an aqueous chondroitin sulfate extract.
10. A method for improving a taste of a preparation for oral administration comprising quercetin glycoside, the method comprising adding an aqueous chondroitin sulfate extract.

### Advantageous Effects of Invention

The present invention enables the bad taste peculiar to quercetin glycoside to be effectively suppressed by adding an aqueous chondroitin sulfate extract. Further, since the preparation for oral administration according to the present invention suppresses the bad taste derived from quercetin glycoside, continuous ingestion becomes easy.

### DESCRIPTION OF EMBODIMENTS

The preparation for oral administration according to the present invention contains an aqueous chondroitin sulfate extract and quercetin glycoside, and can be used in pharmaceutical applications such as veterinary applications, food applications including supplements, cosmetic and beauty applications and the like.

(Chondroitin Sulfate)

The preparation according to the present invention contains chondroitin sulfate obtained by aqueous extraction. Chondroitin sulfate is widely distributed throughout the human body. The joint cartilages and the skin contain an especially large amount of chondroitin sulfate. Consequently, chondroitin sulfate is utilized in health foods to improve osteoarthritis and to make the skin more beautiful. As is understood in the present technical field, in the present invention, the term “chondroitin sulfate” means chondroitin sulfate and/or a salt thereof. Although chondroitin sulfate is contained in the cartilages and the skin, the cartilages contain an especially high concentration of chondroitin sulfate. Along with collagen, chondroitin sulfate serves as a main component to fulfill the function of the cartilages. Further, chondroitin sulfate has a function for retaining moisture and keeping it in the tissues and a function for transporting substances from the tissues. Thus, chondroitin sulfate is an important substance involved in elasticity and moisture retention. Especially, since the amount of chondroitin sulfate in the body is said to decrease with age, its deficiency must be supplemented for normal metabolism from outside of the body by various methods.

As described above, in the present invention, “chondroitin sulfate” means a chondroitin sulfate or salt thereof and includes various chondroitin sulfates. Generally, chondroitin sulfate has a structure in which D-glucuronic acid (uronic acid) and N-acetyl-D-galactosamine are linked, and a sulfonic acid group is bound to this structure. Based on the binding site of the sulfonic acid group, chondroitin sulfate is classified into a plurality of types, such as A, B, C, D, E, and K. Chondroitin sulfate B has L-iduronic acid as a constituent sugar. The chondroitin sulfate used in the present invention may be any of these types. Further, in the present invention, a plurality of the above-described various types may be used in combination, or a single type thereof may be used alone.

Any chondroitin sulfate obtained by aqueous extraction may be used in the present invention. The source of the chondroitin sulfate is not especially limited. In the present invention, chondroitin sulfate can be obtained from shark, squid, salmon, crab, cattle, pig, bird and the like, which contain a large amount of chondroitin sulfate in their cartilages and skin. Generally, chondroitin sulfate A is present in large amounts in higher vertebrates, such as cattle and pigs, and chondroitin sulfate C is present in large amounts in lower vertebrates. Further, cartilaginous fish such as sharks have a high proportion of cartilages so that large amounts of chondroitin sulfate A, C, D and the like can be obtained from the cartilages such as their fins and midrib. Consequently, cartilaginous fish are a preferred raw material for the chondroitin sulfate in the present invention. In addition, chondroitin sulfate E is present in large amounts in squid, and chondroitin sulfate K is present in large amounts in helmet crabs. In the present invention, the source of the chondroitin sulfate can be selected based on the application.

In the present invention, it is preferred to use shark cartilage as a raw material, since chondroitin sulfate is contained in a high concentration. The type of shark used as a raw material for the chondroitin sulfate according to the present invention is not limited. Examples may include Squalus acanthias and Prionace glauca. In addition, the site from which the cartilage is derived is not limited. Examples may include the head, fins and the like.

In the present invention, chondroitin sulfate obtained by aqueous extraction is used. The term “aqueous chondroitin sulfate extract” in the present invention means chondroitin sulfate extracted from a raw material using a water-based solvent. As is described below, although quercetin glycoside has a unique bitter taste, the present inventors discovered that especially adding an aqueous chondroitin sulfate extract effectively suppresses the bad taste of quercetin glycoside, thereby completing the present invention. In the present invention, although the mechanism in which the bad taste of quercetin glycoside is effectively suppressed by an aqueous chondroitin sulfate extract is not clear, it is thought that chondroitin sulfate that has undergone an aqueous extraction step has higher hygroscopicity, a higher dispersion rate in water, and higher adsorption properties than a crushed product obtained by just crushing a raw material such as shark cartilage, and that such properties enable an aqueous chondroitin sulfate extract to effectively suppress the bad taste derived from quercetin glycoside. However, the present invention is not to be limited to the above theory.

Generally, chondroitin sulfate is used as a crushed product obtained by directly crushing a raw material or as an extract obtained by extraction from the raw material. However, in the present invention, chondroitin sulfate obtained by extraction with water can be used. In the present invention, the aqueous chondroitin sulfate extract is not especially limited, as long as it is extracted from the raw material using a water-based solvent. Various chondroitin sulfates may be used. Examples of the water-based solvent include water at various temperatures (hot water, warm water, ordinary temperature water), and water containing a polar solvent such as ethanol and methanol. For example, the aqueous chondroitin sulfate extract in the present invention can be obtained by finely shredding a cartilage such as a shark cartilage to an appropriate size, then performing an extraction treatment in the water-based solvent, purifying the resultant product by a method such as adsorption or filtration, adding an excipient such as dextrin, and forming a powder by a method such as spray drying. A proteolytic enzyme and the like can be added during the aqueous extraction. Further, hot water can be used as the solvent.

In the present invention, as the aqueous chondroitin sulfate extract, either a complex (protein complex) formed from chondroitin sulfate and a protein may be used, or chondroitin sulfate (protein-removed product) from which protein has been removed may be used.

Since protein complexes tend to maintain their structure in a living organism to some extent, and are easily integrated and used in the body, they tend to be preferred as a food ingredient. In the present invention, a protein complex can be preferably used. Examples of commercially-available products in which a protein complex is used include SCP (manufactured by Maruha Nichiro Foods, Inc.).

On the other hand, a protein-removed product can be used which has undergone a protein removing step, such as alcohol purification in a purification step. Examples of commercially-available products include Chondroitin Q (manufactured by Kewpie Corporation). Further, usually, sodium chondroitin sulfate, which is often used in pharmaceuticals and cosmetic applications, may also be used.

In the preparation according to the present invention, the added amount of chondroitin sulfate can be determined based on a guide of preferably 10 to 3,000 mg/day, and more preferably 50 to 1,000 mg/day, per individual. Further, the intake per 1 kg of body weight can be set to, for example, 0.15 to 50.0 mg/kg, and more preferably 0.80 to 20.0 mg/kg. In addition, the added ratio of chondroitin sulfate in the oral preparation is preferably 0.1 to 95 wt. %, and more preferably 0.5 to 20 wt. %, based on the whole preparation.

(Quercetin Glycoside)

The solid preparation according to the present invention contains quercetin glycoside. As described below, quercetin glycoside is said to have an anti-inflammatory effect. It is thought that joint pain and other such pain can be more effectively reduced by ingesting quercetin glycoside simultaneously with chondroitin sulfate. The quercetin glycoside in the present invention is a glycoside of quercetin (in Japanese, also called “kuerusechin”), which is one of flavonoids. Examples thereof include rutin, quercitrin, isoquercitrin, morin, myricitrin, myricetin, hesperidin, naringin, and tangerijin. Further, “quercetin glycoside” also includes an enzyme-treated product of such quercetin glycosides.

The quercetin glycoside used in the present invention is known to exhibit various physiological effects. Quercetin glycoside has been reported to have an anti-inflammatory effect, an antioxidant effect, a vasoconstriction effect, a capillary permeation inhibition effect, and a capillary wall strengthening effect, and has also been reported to inhibit several enzymes relating to cell growth. However, quercetin glycoside has a unique taste, especially a bitter taste, and thus oral ingestion is difficult on a daily basis as a food. Therefore, in the preparation for oral administration according to the present invention, a preparation for oral administration having an improved taste is obtained by adding the above-described aqueous chondroitin sulfate extract to quercetin glycoside.

As the quercetin glycoside used in the present invention, rutin or an analog thereof is preferred. Rutin is one of flavonoids that is classified as a vitamin-like substance due to its vitamin-like action. Rutin can typically be obtained not only from Rutaceous plants, but also from the Chinese scholar tree from the Fabaceae family and buckwheat from the Polygonaceae family.

The origin and the production method of the quercetin glycoside used in the present invention are not especially limited. Examples of plants known to contain a large amount of quercetin glycoside include capers, apples, tea, onions, grapes, broccoli, mulukhiyah, raspberries, lingonberries, cranberries, Opuntia, leafy vegetables, and citrus. The quercetin glycoside may be obtained from these plants.

In a preferred embodiment of the present invention, quercetin glycoside which has been transglycosylated by treating the quercetin glycoside with glycosyltransferase, for example, can be used. Although a quercetin glycoside such as rutin can be difficult to use due to its poor solubility in water, because an enzyme-treated product has higher water solubility due to transglycosylation, quercetin glycoside may be suitably used in the preparation according to the present invention.

In an especially preferred embodiment according to the present invention, an enzyme-treated product of rutin (hereinafter, “enzyme-treated rutin”) is used as the quercetin glycoside. Enzyme-treated rutin, which is also called “enzyme-treated isoquercitrin” and “transglycosylated rutin”, refers to a quercetin glycoside having α-glycosyl isoquercitrin that has been transglycosylated by subjecting rutin or an analog thereof to an enzyme treatment as a main component. Generally, although rutin is known to have an antioxidant effect, the applications in which it could be used were limited due to its poor solubility in water. However, solubility in water can be increased by subjecting enzyme-treated rutin to transglycosylation. Such a process is preferred. In addition to having a strong antioxidative activity, enzyme-treated rutin is known to have a large variety of physiological functions, such as platelet aggregation inhibition and adherence inhibition effects, a vasodilatory effect, and an anticancer effect. Accordingly, enzyme-treated rutin is utilized in health foods directed to effects such as improving inflammation and promotion of blood circulation. Enzyme-treated rutin can be obtained by, for example, treating an extract of the Chinese scholar tree, buckwheat and the like with glycosyltransferase. Specifically, enzyme-treated rutin can be obtained by the method described in Japanese Patent Laid-Open No. 7-10898.

The added amount of the quercetin glycoside in the preparation according to the present invention can be determined so that the enzyme-treated rutin intake is, as a guide, 1 to 500 mg/day, and preferably 5 to 300 mg/day, per individual. Further, the intake per 1 kg of body weight can be set to, for example, 0.015 to 8.5 mg/kg, and more preferably 0.080 to 5.0 mg/kg. In addition, the added ratio of quercetin glycoside in the oral preparation can be set to 0.1 to 95 wt. %, and preferably 0.5 to 80 wt. %, based on the whole preparation.

In the preparation according to the present invention, the weight ratio of the aqueous chondroitin sulfate extract and the quercetin glycoside is preferably 1:50 to 200:1, more preferably 1:5 to 50:1, and most preferably 1:5 to 20:1.

(Glucosamine)

In a preferred embodiment, the preparation according to the present invention contains glucosamine. Adding glucosamine to chondroitin sulfate is said to be good (G. C. Reyes, et al., Progress in Drug Res., 55, pp. 83-103, 2000), and thus it is preferred to add glucosamine to the preparation according to the present invention. Glucosamine is 2-aminoglucose in which the hydroxyl group at position 2 of glucose is substituted with an amino group. Glucosamine is a natural amino sugar widely distributed in the natural world as a constituent sugar molecule of glycoproteins, glycolipids, and mucopolysaccharides, which are biological components. Glucosamine can be industrially obtained by hydrolyzing chitin included in crustaceans, such as crabs, shrimp, and krill, and the cartilage from squid, with an acid or an enzyme, and then separating and purifying the resultant product.

Recently, not only the importance of glucosamine as a basic structural molecule in biological components, but also various efficacies resulting from the ingestion of glucosamine have been confirmed. Consequently, glucosamine is widely used in health foods for its effects in the treatment and prevention of joint pain and osteoarthritis, and for beauty. In the preparation according to the present invention, in addition to chondroitin sulfate and quercetin glycoside, it is preferred to add glucosamine to obtain an expected alleviation of joint pain.

The origin and the production method of the glucosamine used in the present invention are not especially limited. Further, the glucosamine may be used as a salt or various derivatives, and the type of these salts or derivatives is not especially limited. When the term “glucosamine” is used just by itself in the present invention, the term includes a salt or derivative thereof. Examples of a salt or derivative of glucosamine include glucosamine hydrochloride, glucosamine sulfate, glucosamine lactate, and N-acetylglucosamine.

The added amount of glucosamine in the preparation can be determined so that the glucosamine intake is, as a guide, 100 to 5,000 mg/day, and preferably 300 to 2,000 mg/day, per individual. Further, the intake per 1 kg of body weight can be set to, for example, 1.5 to 85.0 mg/kg, and more preferably 5.0 to 60.0 mg/kg. For example, the added ratio of glucosamine in a tablet can be set to 0.1 to 95 wt. %, and preferably 10 to 80 wt. %, based on the whole tablet.

(Other Components etc.)

In addition to the aqueous chondroitin sulfate extract and quercetin glycoside, the preparation according to the present invention may also contain an ingredient having some other biofunction, such as collagen, hyaluronic acid, and vitamins. Further, the preparation according to the present invention may also contain an additive and an auxiliary ingredient usually used in foods and pharmaceuticals, such as a sweetener, an acidulant, an excipient, a lubricant, a flavoring agent such as lemon flavoring or a milk flavoring, starch, and dextrin.

The present invention is an oral preparation, which is especially suitable as a preparation for oral administration due to the fact that the bad taste derived from quercetin glycoside is suppressed by an aqueous chondroitin sulfate extract. The preparation according to the present invention is preferably used in a form suited to oral administration, such as a tablet, a capsule, granules, a powder, a lozenge, or in liquid dosage form, such as a drink. Among these, from the perspective of including quercetin glycoside, which is the component causing the bad taste, the preparation according to the present invention is preferably a tablet or granules. Further, from the perspective of using an aqueous chondroitin extract for the chondroitin sulfate, a liquid preparation is also preferred.

In a preferred embodiment, the preparation according to the present invention can be prepared as a tablet having a weight of 50 mg to 5,000 mg, and preferably 70 mg to 1,000 mg, per tablet. Further, in the case of granules, a sachet can be prepared so that it has a weight of 50 mg to 5,000 mg, and preferably 300 mg to 3,000 mg of granules per sachet. In the case of a container/package such as a bottle, the container/package can be prepared so that it contains 5 g to 5 kg, for example, of granules per container/package. In addition, if the preparation according to the present invention is a liquid preparation, such as a drink, it can be prepared as a bottled product containing 5 g to 2 kg, for example.

The administration target of the preparation according to the present invention is not especially limited, and may be a human or an animal other than a human. The dosage form can be appropriately selected based on the administration target. For example, if a human is the administration target, it may be difficult for middle-aged and elderly people having a high risk of the onset of arthritis to swallow a tablet. Therefore, for such people, it is especially preferable to use the preparation according to the present invention as granules or a chewable tablet. Further, if a child is the administration target, the preparation according to the present invention may be used as a sugar-coated tablet. In addition, if an animal other than a human is the administration target, because palatability has a large influence on how easily the preparation can be administered, the preparation according to the present invention with its bad taste of quercetin glycoside improved is suited to animal administration. For example, for pets (especially, dogs and cats) that tend to chew on a tablet in their mouth, it is especially preferred to select a chewable tablet as the dosage form.

The preparation according to the present invention can be used as a food, a pharmaceutical, a cosmetic, and a food additive. Since the bitter taste derived from quercetin glycoside is suppressed, the preparation according to the present invention can be ingested on a daily basis without problem, and is suited to use as a food such as a so-called “functional food”.

(Method)

Further, from a different perspective, the present invention can be recognized as a method for producing a preparation for oral administration containing quercetin glycoside, which includes the step of adding an aqueous chondroitin sulfate extract. From still another perspective, the present invention can be understood as a method for improving the taste of a preparation for oral administration containing quercetin glycoside, which includes the step of adding an aqueous chondroitin sulfate extract.

### EXAMPLES

The present invention will now be described in more detail with reference to the following Examples. However, the present invention is not limited to the following Examples. Further, unless stated otherwise, in the present invention, “parts”, “%” and the like are in terms of weight, and numerical ranges include the end points thereof.

### Example 1

Compositions containing quercetin glycoside were investigated as to what blend suitably masked the bad taste of quercetin glycoside. Specifically, an aqueous chondroitin sulfate extract (a protein complex and a protein-removed product), crushed chondroitin sulfate, and dextrin were investigated as components for masking the bad taste of quercetin glycoside.

(Materials)

The materials used are as follows.

Quercetin glycoside: San Emic P15 (manufactured by San-Ei Gen F.F.I., Inc., quercetin glycoside content 15%)

Aqueous chondroitin sulfate extract (protein complex): SCP (manufactured by Maruha Nichiro Foods, Inc., chondroitin sulfate content 20%, powder formed by adding dextrin to a hot-water extract of shark cartilage)

Aqueous chondroitin sulfate extract (protein-removed product): Chondroitin Q (manufactured by Kewpie Corporation, chondroitin sulfate content 20%, powder formed by purifying a hot-water extract of shark cartilage with alcohol to remove complex protein, and then adding dextrin)

Crushed chondroitin sulfate: Shark Cartilage Powder KSCP-S (manufactured by Nippon Suisan Kaisha, Ltd., chondroitin sulfate content 20%, crushed powder of shark cartilage)

Dextrin: Sandek #250 (manufactured by Sanwa Cornstarch Co., Ltd.)

### (Evaluation Method)

Mixed powders were prepared by blending the various masking components with quercetin glycoside in the blends shown in Table 1. Six panelists were orally administered with 1 g of the prepared powders with water. The six panelists evaluated the strength of the bitter taste caused by quercetin glycoside based on the following criteria.

Three points: “Bitter”

Two points: “Slightly bitter”

One point: “Sensed a weak bitter taste”

Zero points: “Hardly sensed any bitter taste”

The evaluations from the six panelists were collated. Samples having an average score of more than 2 points to 3 points or less were evaluated with a “X”, samples having an average score of more than 1 point to 2 points or less were evaluated with a “Δ”, and samples having an average score of 0 points to 1 point or less were evaluated with a “◯”. The results are shown in Table 1.

TABLE 1
Quercetin glycoside bitter taste suppression

| | | Sample No. | | | | | | | | | | | | | | | | | | | |
|---|---|---|---|---|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 | 16 | 17 | 18 | 19 | 20 |
| Quercetin glycoside | | 10 | 10 | 10 | 1 | 0.1 | 10 | 10 | 10 | 1 | 0.1 | 10 | 10 | 10 | 1 | 0.1 | 10 | 10 | 10 | 1 | 0.1 |
| Masking | Aqueous | 0.15 | 1.5 | 7.5 | 15 | 15 | | | | | | | | | | | | | | | |
| component | chondroitin | | | | | | | | | | | | | | | | | | | | |
| | sulfate extract | | | | | | | | | | | | | | | | | | | | |
| | (Protein complex) | | | | | | | | | | | | | | | | | | | | |
| | Aqueous | | | | | | 0.15 | 1.5 | 7.5 | 15 | 15 | | | | | | | | | | |
| | chondroitin | | | | | | | | | | | | | | | | | | | | |
| | sulfate extract | | | | | | | | | | | | | | | | | | | | |
| | (Protein-removed | | | | | | | | | | | | | | | | | | | | |
| | product) | | | | | | | | | | | | | | | | | | | | |
| | Crushed chondroitin | | | | | | | | | | | 0.15 | 1.5 | 7.5 | 15 | 15 | | | | | |
| | sulfate | | | | | | | | | | | | | | | | | | | | |
| | Dextrin | | | | | | | | | | | | | | | | 0.15 | 1.5 | 7.5 | 15 | 15 |

| | | | | | | | | | | | | | | | | | | | | |
|---|---|---|---|---|---|---|---|---|---|---|---|---|---|---|---|---|---|---|---|---|
| Mixing ratio of masking | 1/50 | ⅕ | 1 | 20 | 200 | 1/50 | ⅕ | 1 | 20 | 200 | 1/50 | ⅕ | 1 | 20 | 200 | 1/50 | ⅕ | 1 | 20 | 200 |
| component/Quercetin glycoside | | | | | | | | | | | | | | | | | | | | |
| Sensory evaluation result | Δ | ◯ | ◯ | ◯ | ◯ | Δ | ◯ | ◯ | ◯ | ◯ | X | Δ | Δ | Δ | ◯ | X | X | Δ | Δ | ◯ |

It is clear from the results shown in Table 1 that the bitter taste of quercetin glycoside could be effectively masked by chondroitin sulfate obtained by aqueous extraction.

### Example 2

### Production and Evaluation of Solid Preparations

Compositions containing quercetin glycoside and various masking components were formed into preparations, and the masking effect on the bad taste of the quercetin glycoside was evaluated. The materials used are as follows.

Quercetin glycoside: San Emic P15 (manufactured by San-Ei Gen F.F.I., Inc., quercetin glycoside content 15%)

Aqueous chondroitin sulfate extract (protein complex): SCP (manufactured by Maruha Nichiro Foods, Inc., chondroitin sulfate content 20%, powder formed by adding dextrin to a hot-water extract of shark cartilage)

Aqueous chondroitin sulfate extract (protein-removed product): Chondroitin Q (manufactured by Kewpie Corporation, chondroitin sulfate content 20%, powder formed by purifying a hot-water extract of shark cartilage with alcohol to remove complex protein, and then adding dextrin)

Crushed chondroitin sulfate: Shark Cartilage Powder KSCP-S (manufactured by Nippon Suisan Kaisha, Ltd., chondroitin sulfate content 20%, crushed powder of shark cartilage)

### (Tablets)

About 3 kg of a mixed powder was prepared by mixing raw material powders according to the blends shown in Table 2. The mixing of the respective components was carried out by charging the raw material powders into a mixer (Kotobuki Mixwell V-100, manufactured by Tokuju Corporation) and mixing for 5 minutes at 22 rpm. The obtained mixed powders were subjected to direct tableting using a mortar and pestle (HT-AP15SS-II, manufactured by Hata Iron Works Co., Ltd.) at a pounding pressure of about 2,000 kgf and at a rotation speed of about 20 rpm with a diameter of 10 mm to obtain tablets 9 mm in diameter and having a weight of 370 mg per tablet.

Six panelists were asked to orally ingest one tablet, and after keeping the tablet in their mouth for 10 seconds, evaluate the taste when swallowing the tablet based on the following criteria.

Three points: “Bitter”

Two points: “Slightly bitter”

One point: “Sensed a weak bitter taste”

Zero points: “Hardly sensed any bitter taste”

The evaluations from the six panelists were collated. Samples having an average score of more than 2 points to 3 points or less were evaluated with a “X”, samples having an average score of more than 1 point to 2 points or less were evaluated with a “Δ”, and samples having an average score of 0 points to 1 point or less were evaluated with a “◯”. The results are shown in Table 2.

TABLE 2
Tablets

| | Sample 1 | Sample 2 | Sample 3 |
|---|---|---|---|
| Quercetin glycoside | 15 | 15 | 15 |
| Aqueous chondroitin sulfate | 15 | 0 | 0 |
| extract (Protein complex) | | | |
| Aqueous chondroitin sulfate | 0 | 15 | 0 |
| extract (Protein-removed product) | | | |
| Crushed chondroitin sulfate | 0 | 0 | 15 |
| Glucosamine hydrochloride | 50 | 50 | 50 |
| Cellulose | 15 | 15 | 15 |
| Silicon oxide | 1.5 | 1.5 | 1.5 |
| Sucrose fatty acid ester | 3.5 | 3.5 | 3.5 |
| Powder weight (%) Total | 100 | 100 | 100 |
| Sensory evaluation result | ◯ | ◯ | Δ |

As shown in Table 2, the masking effects of an aqueous chondroitin sulfate extract against the bitter taste of quercetin glycoside were confirmed.

(Granules)

About 3 kg of a mixed powder was prepared by mixing raw material powders according to the blends shown in Table 3. As a binder, 600 g of an aqueous 60% ethanol solution was prepared and charged into the mixed powders. The resultant mixtures were kneaded using a kneader (HU-N, manufactured by Hata Iron Works Co., Ltd.), and then granulated using an extrusion granulating machine (HU-G, manufactured by Hata Iron Works Co., Ltd.). A screen having 1.0 mm apertures was used. The granulated product was dried for 30 minutes at 60° C. with a dryer (MOV-112F, manufactured by Sanyo Electric Co., Ltd.), and then sieved using a No. 16 sieve (1,000 μm apertures). The product was further sieved using a No. 80 sieve (117 μm apertures). The fine particles that had passed through the sieve were removed, to thereby obtain the granules.

Six panelists were asked to orally ingest 1 g of the prepared granules, and after keeping the granules in their mouth for 10 seconds, evaluate the taste when swallowing the granules based on the following criteria.

Three points: “Bitter”

Two points: “Slightly bitter”

One point: “Sensed a weak bitter taste”

Zero points: “Hardly sensed any bitter taste”

The evaluations from the six panelists were collated. Samples having an average score of more than 2 points to 3 points or less were evaluated with a “X”, samples having an average score of more than 1 point to 2 points or less were evaluated with a “Δ”, and samples having an average score of 0 points to 1 point or less were evaluated with a “◯”. The results are shown in Table 3.

TABLE 3
Granules

| | Sample 1 | Sample 2 | Sample 3 |
|---|---|---|---|
| Quercetin glycoside | 15 | 15 | 15 |
| Aqueous chondroitin sulfate | 15 | 0 | 0 |
| extract (Protein complex) | | | |
| Aqueous chondroitin sulfate | 0 | 15 | 0 |
| extract (Protein-removed product) | | | |
| Crushed chondroitin sulfate | 0 | 0 | 15 |
| Glucosamine hydrochloride | 50 | 50 | 50 |
| Starch | 20 | 20 | 20 |
| Powder weight (%) Total | 100 | 100 | 100 |
| Sensory evaluation result | ◯ | ◯ | Δ |

As shown in Table 3, the masking effects of an aqueous chondroitin sulfate extract against the bitter taste of quercetin glycoside were confirmed.

(Pet Tablets)

About 3 kg of a mixed powder was prepared by mixing raw material powders according to the blends shown in Table 4. The mixing of the respective components was carried out by charging the raw material powders into a mixer (Kotobuki Mixwell V-100, manufactured by Tokuju Corporation) and mixing for 5 minutes at 22 rpm. The obtained mixed powders were subjected to direct tableting using a mortar and pestle (HT-AP15SS-II, manufactured by Hata Iron Works Co., Ltd.) at a pounding pressure of about 2,000 kgf and at a rotation speed of about 20 rpm with a diameter of 10 mm to obtain tablets 11 mm in diameter and having a weight of 500 mg per tablet.

TABLE 4
Pet tablets

| | Sample 1 | Sample 2 |
|---|---|---|
| Quercetin glycoside | 15 | 15 |
| Aqueous chondroitin sulfate | 15 | 0 |
| extract (Protein complex) | | |
| Aqueous chondroitin sulfate | 0 | 15 |
| extract (Protein-removed product) | | |
| Crushed chondroitin sulfate | 0 | 0 |
| Glucosamine hydrochloride | 50 | 50 |
| Cellulose | 15 | 15 |
| Silicon oxide | 1.5 | 1.5 |
| Sucrose fatty acid ester | 3.5 | 3.5 |
| Powder weight (%) Total | 100 | 100 |

Thus, pet tablets that masked the bitter taste of quercetin glycoside with aqueous chondroitin sulfate extract were obtained.

### Example 3

### Drink Production

Weighed were 0.5 g of quercetin glycoside in terms of active component and 0.5 g of aqueous chondroitin sulfate extract (protein-removed product) in terms of active component. In addition, weighed were 10 g of N-acetylglucosamine, 1.2 g of sodium ascorbate, 26.5 g of sucrose, 2.1 g of citric acid, and 0.3 mg of anhydrous caffeine. These raw materials were dissolved by adding water to form a 1,000 g mixture. This mixture was injected as 100-g-portions into brown bottles. The bottles were subjected to retort sterilization to obtain the drink according to the present invention.

1. A preparation for oral administration, comprising quercetin glycoside and a water-based solvent extract of cartilage containing chondroitin sulfate.

2. The preparation for oral administration according to claim 1, wherein the cartilage is fish cartilage.

3. The preparation for oral administration according to claim 2, wherein the fish cartilage is shark cartilage.

4. The preparation for oral administration according to any claim 1, wherein the quercetin glycoside is enzyme-treated rutin.

5. The preparation for oral administration according to claim 1, further comprising glucosamine.

6. The preparation for oral administration according to claim 1, wherein the preparation is a solid preparation.

7. The preparation for oral administration according to claim 6, wherein the solid preparation is a tablet or a granular form.

8. The preparation for oral administration according to claim 1, wherein a weight ratio of the water-based solvent extract of cartilage containing chondroitin sulfate and the quercetin glycoside is 1:50 to 200:1.

9. The preparation for oral administration according to claim 1, wherein the chondroitin sulfate exists as a complex with a protein.

10. The preparation for oral administration according to claim 1, wherein the preparation is a functional food.

11. A method for producing a preparation for oral administration comprising quercetin glycoside, the method comprising blending quercetin glycoside with a water-based solvent extract of cartilage containing chondroitin sulfate.

12. A method for improving a taste of a composition comprising quercetin glycoside, the method comprising adding a water-based solvent extract of cartilage containing chondroitin sulfate.
